(19) United States
(12) Patent Application Publication
(10) Pub. No.: US 2008/0227220 A1
(43) Pub. Date: Sep. 18, 2008

(54) Lateral Flow Binding Assay

(76) Inventors: Maartje-Maria Franse, Delft (NL); IJSbrand Johan Blankwater, Schagen (NL); Michael Valentine, Portland, ME (US)

Correspondence Address: NIXON & VANDERHYE, PC, 901 NORTH GLEBE ROAD, 11TH FLOOR, ARLINGTON, VA, 22203, US

(21) Appl. No.: 10/586,617

(22) PCT Filed: Jan. 26, 2005
(86) PCT No.: PCT/EP05/00837

§ 371 Date: Jul. 19, 2006

(30) Foreign Application Priority Data
Jan. 29, 2004 (EP) 04075266.9

(51) Int. Cl. G01N 33/536; B01J 19/00

(52) U.S. Cl. 436/536; 422/68.1

(57) ABSTRACT

Provided is a test device, a test method and a test kit for the detection of the presence or absence of an analyte in a fluid sample, based on a device comprising a backing with a first end (A) and a second end (B) and consecutively going from first end (A) to second end (B): (a) a sample receiving section attached to one side of the backing; (b) an analyte detection section attached to said one side of the backing comprising a capture site; (c) an absorption section attached to said one side of the backing; (d) a reaction section; wherein the sample receiving section is in fluid contact with the analyte detection section, which is in fluid contact with the absorption section and wherein there is no fluid contact between the reaction section and any of the other sections.

### FIELD OF THE INVENTION

The present invention relates to an improved novel lateral flow binding assay device and a method for the rapid determination of the presence, absence or amount of an analyte in a fluid.

### BACKGROUND OF THE INVENTION

Lateral flow binding assays are used for the detection of analytes in fluids. The analytes to be tested can be of various origins, for example antibiotics, bacteria, carbohydrates and hormones. A well-known example of such an assay is the pregnancy test. Other examples can be found in the area of antibiotic assays, such as meat, milk or urine tests.

In general, the assays based on lateral flow binding technology are typified by the binding reaction between an antigen and its complementary antibody; such assays are also known as immunochromatographic assays. However, the term lateral flow binding assay also encompasses assays that are based on the recognition and/or binding of an analyte to any suitable binding particle which can be either natural or non-natural, for example a protein suitable for binding, not necessarily an antibody.

Lateral flow binding assay devices generally comprise a fluid sample receiving section, an analyte detection section and an absorption section that are all attached to one side of an essentially flat surface, such as a strip, usually made of inert material such as glass, metal or preferably a plastic.

The principle of this assay is visualization of the binding between analyte and a suitable binding particle in the analyte detection section. To this end, a label can be attached to the suitable binding particle. Examples of compounds that are used as label are compounds that produce a visual signal in the analyte detection section, such as dyes (i.e. chromogenic or fluorescent dyes), certain metal particles (i.e. gold sots), other colored particles (i.e. latex particles), or particles based on other means of detection, such as radioactive compounds. In order to achieve visualization, the analyte detection section contains a capture site where the complex between analyte and the suitable binding particle is retained, for instance by means of an immobilized antigen. Also, there are many examples wherein more than one caption sites with different functionalities are present in the analyte detection section.

Alternatively, the assay may be designed in such a way that visualization only occurs when there is no binding between analyte and the suitable binding particle, i.e. in the case when there is no analyte present in the sample.

As outlined above, an essential step in lateral flow binding assay technology is contacting the sample to be analyzed with a suitable binding particle to which in most cases a label is attached.

This may be achieved by placing the suitable binding particle in a container in which the sample is to be placed. Afterwards, an assay strip as described above is placed in the container. An example of this type of assay device is described in International Patent Applications WO 99/18439 and WO 99/67416. The disadvantage of this system is the fact that the assay device comprises at least two components, i.e. the assay strip and the container with the suitable binding particle. Moreover, many of the commercially available devices that operate according to this principle require the presence of a housing of any kind. Altogether, this gives rise to a complex manufacturing and packaging procedure and results in a product wherein there is no freedom of operation for the end-user with regard to the type of container to be used. Containers can also not be re-used.

Alternatively, the suitable binding particle is already present on the assay strip and localized in such a way that the sample first passes a section wherein the suitable binding particle is present, i.e. the reaction section, prior to passage of the analyte detection section. Examples of this type of assay device are described in European Patent 0 323 605 B1 and U.S. Pat. No. 5,712,172. The disadvantage of this type of assay device is that there is no, or hardly any, control over the time available for interaction between sample and the suitable binding particle. This may lead to an incomplete reaction between analyte and suitable binding particle, which is particularly disadvantageous for highly sensitive and semi-quantitative assays since reliable results cannot be expected.

The above merely reflects the difficulties encountered with contacting a sample with a suitable binding particle. Similar difficulties also arise in other types of pre-treatment procedures, for instance when cells to be analyzed have to undergo lysis prior to analysis or when a desired pH-value has to be set.

Consequently, there is a need for an improved assay device and assay method that does not have the problems described above.

### SUMMARY OF THE INVENTION

is an object of the present-invention to provide an improved lateral flow binding assay for the determination of an analyte in a fluid. The lateral flow binding assay device of the present invention provides a simple and easy to use device wherein the optimal conditions for performing a pre-treatment step and the step for detection of the analyte can be set independently of each other whilst all necessary components of the device are present on one member. The advantage is that the optimal condition for the one process can be set without comprising that of the other process. Furthermore, the lateral flow binding assay device of the present invention does not require any additional components such as a housing, whereby a considerable simplification of the production process is reached.

Thus, the present invention provides a device for the detection of the presence or absence of an analyte in a fluid sample comprising a backing with a first end (A) and a second end (B) and consecutively going from first end (A) to second end (B):
(a) a sample receiving section attached to one side of the backing;
(b) an analyte detection section attached to said one side of the backing comprising a capture site;
(c) an absorption section attached to said one side of the backing;
(d) a reaction section;
   wherein the sample receiving section is in fluid contact with the analyte detection section, which is in fluid contact with the absorption section and wherein there is no fluid contact between the reaction section and any of the other sections.

Furthermore, the present invention provides a method for detecting an analyte in a fluid sample comprising the steps of:
(a) contacting the reaction section present at second end (B) of the device described above with the fluid sample for a period of at least 10 seconds to 10 minutes;
(b) removing the reaction section present at second end (B) of the device described above from the fluid sample;
(c) contacting the first end (A) of the device described above with the fluid sample for a period of at least 1 to 10 minutes;
(d) detecting the analyte by observing the capture site of the device described above.

Additionally, the present invention provides a kit suitable for the determination of an analyte in a fluid comprising a device as described above and optionally a thermostatic device, with the aid of which test samples can be kept at a pre-set temperature and the use of a device as described above for the determination of the presence or absence of an analyte in a fluid sample

### DETAILED DESCRIPTION OF THE INVENTION

terms and abbreviations given below are used throughout this disclosure and are defined as follows.

‘Absorption section’ refers to the part of the assay device, which is in lateral flow contact with the analyte detection section and functions to promote lateral flow through the analyte detection section and is capable of absorbing excess sample. The contact can be an overlap or an end-to-end connection. The absorption section is made of porous material.

‘Analyte’ refers to a material the presence and/or absence and/or the quantity of which is to be determined in the sample. Examples of analytes are antibiotics, carbohydrates, dietary substances, drugs, hormones, immune-response proteins, microorganisms, (poly)nucleotides, (poly)peptides, steroids, viruses, vitamins and the like.

‘Analyte detection section’ refers to the portion of the assay device which is in lateral flow contact with the porous material of the sample receiving section and the absorption section. The contact can be an overlap or an end-to-end connection. The analyte detection section is made of porous material. The analyte detection section usually comprises one or more capture sites. For instance, it is fairly common to insert a capture site for detection of the presence or absence of the analyte, and a second capture site that functions as a control site.

‘Assay’ refers to the determination of the presence and/or absence and/or the quantity of one or more components of an analyte.

‘Backing’ refers to material that is used to provide support for members. More particular, in the present case said members are for instance a sample receiving section, an analyte detection section, a capture site, an adsorption section, a reaction section and the like. When used for a lateral flow binding assay device, a backing usually is made from material that is inert with respect to the application for which the device is to be used. Suitable materials are glass, metals and various types of plastics. Attachment of the members to the backing can be performed following known techniques such as gluing, thermo compression and the like. For the assay devices of the present invention, a backing usually has a length varying between 2 and 50 cm, preferably between 4 and 25 cm, more preferably between 5 and 10 cm, a width varying between0.1 and-2 cm, preferably between 0.2 and 1 cm, more preferably between 0.3 and 0.5 cm, and a thickness varying between 0.005 and 0.5 cm, preferably between 0.01 and 0.1 cm, more preferably between 0.02 and 0.05 cm.

‘Capture reagent’ refers to any reagent that can be used to create the required functionality in a capture site. A capture reagent may be any natural or non-natural particle that is suitable for binding to the analyte-binding particle complex and/or the binding particle. Examples of suitable capture reagents are antibiotics, antibodies, antigens, ligands. Preferably said capture reagents are designed such that upon contact with the capture site they will bond to the capture site, either covalently or by means of other bonding principles.

‘Capture site’ refers to a defined area preferably located within the analyte detection section. The capture site may be made of a porous material different than that of the analyte detection section. Preferably the capture site is of the same material as the analyte detection section. Most preferably the capture site is made by applying the appropriate capture reagent or mixture of capture reagents to the analyte detection section, either by means of covalent linkages or other bonding processes. The application of the capture reagent to the capture site can be done by known methods such as spraying, painting, drawing, printing, striping and the like. The capture site is capable of generating a signal, for instance a visual color signal, a fluorescent signal or a radioactive signal upon presence or absence of the complex between analyte and suitable binding particle.

‘Fluid’ refers to a substance (as a liquid) tending to flow or conform to the outline of its container.

‘Fluid contact’ refers to the contact between sections in such a manner that fluid sample can flow from one section to the other.

‘Labeling reagent’ refers to any particle, protein or molecule, either natural or non-natural, which recognizes or binds to the analyte to be detected in the sample. Examples are one or more antibodies and/or suitable binding particles such as receptors like, for instance, penicillin binding protein. The labeling reagent has attached to it, by conjugation, covalent bonding or non-covalent bonding any substance that is capable of producing a signal that is detectable by visual or instrumental means. Examples of such substances are catalysts, chromogens, colloidal metallic and non-metallic compounds, dyes, enzymes, fluorescent compounds, latex particles, liposomes comprising signal producing compounds and the like.

‘Lateral flow’ refers to liquid flow in a material in which all of the dissolved and/or dispersed components of the sample are transported at essentially equal velocities and with relatively unimpaired flow laterally through the material.

‘Porous material’ refers to any material capable of providing lateral flow. Examples of suitable porous materials are acrylonitrile copolymer, cotton, glass fiber, nitrocellulose, nitrocellulose blends with polyester or cellulose, nylon, paper, rayon and the like.

‘Pre-treatment compound’ refers to any compound or mixture of compounds that is used for pre-treating a sample. For instance, a pre-treatment compound may be a suitable binding particle. Also, a pre-treatment compound may be a compound with buffering capacity that is added to the reaction section in order to adjust the pH to a desired value. Finally, a pre-treatment compound may also be a compound that is suitable for realizing certain biochemical or chemical reactions. Examples of such reactions are cell-lysis, formation of complexes, solubilization of complexes and the like. Mixtures of the compounds mentioned above may also be introduced as pre-treatment compounds.

‘Reaction section’ refers to the portion of the assay device, which is brought into direct contact with the sample prior to contacting the sample with the sample receiving section. The reaction section may comprise a labeling reagent and/or a pre-treatment compound as defined above. The reaction section can be made of porous material. However, the reaction may also be made of material that partly or fully dissolves in the sample. Compounds facilitating dissolution and/or mixing of the labeling reagent and the sample can be added to the reaction section.

‘Sample’ refers to any biological or synthetic fluid that may contain an analyte for detection. Examples of suitable samples are aqueous solutions, blood, fruit juice, meat juice, milk, urine, waste-water and the like.

‘Sample receiving section’ refers to the portion of the assay device, which is brought into direct contact with the sample after the sample has been contacted with a suitable binding particle. The sample receiving section may comprise a labeling reagent, particularly when such a labeling reagent is not present the reaction section. The sample receiving section is made of porous material.

‘Sensitivity’ refers to the degree of receptiveness of a given system to sense a certain state. More particularly, in the present case ‘sensitivity’ refers to the degree by which concentrations of analytes in a sample can be determined.

‘Threshold’ refers to the concentration value above which a given analyte is to be regarded as present and below which said analyte is to be regarded as absent. Generally, a threshold value is given for particular analytes in particular samples by local, regional or interregional authorities but it can also be pre-set for certain research purposes.

In a first aspect of the invention there is provided a device for the detection of the presence or absence of an analyte in a fluid sample. The device comprises a backing with a first end (A) and a second end (B). Going from (A) to (B), the following sections are attached to the backing. A sample receiving section as defined above, an analyte detection section as defined above, an absorption section as defined above, and a reaction section as defined above. The sample receiving section may be a separate entity however also part of the analyte detection section may serve as sample receiving section. All sections, with the exception of the reaction section, are in fluid contact with the one(s) next to them, for instance by means of overlap or an end-to-end connection. All sections, with the exception of the reaction section, are placed on one side of the backing. The reaction section may also be located on said one side of the backing but this is not an absolute requirement. Preferably, there is no fluid contact between the reaction section and any of the other sections.

In one embodiment of the first aspect of the present invention, the reaction section comprises one or more labeling reagents as defined above. Depending on the set-up of the device, the labeling reagent does or does not bind to the analyte. The labeling reagent may be a suitable binding particle such as, for instance, a penicillin binding protein to which a substance is attached that is capable of producing a signal such as, for instance, a latex or gold particle. If the analyte is present it will bind to the suitable binding particle to form an analyte-protein-label complex.

In another embodiment of the first aspect of the present invention, the reaction section is located on the side opposite to the side of the backing where sample receiving section, analyte detection section and absorption section are located. This has the advantage that, when the reaction section is made of a material that easily leaks sample fluid, this leakage fluid will not contact the absorption section when the device is turned upside down as outlined in the second aspect of the invention. Alternatively, the reaction section is located on both sides of the backing or fully encompasses second end (B) of the backing.

In still another embodiment of the first aspect of the present invention, the reaction section comprises a pre-treatment compound as defined above.

The person skilled in the art will appropriately combine the possibilities as set out in the above embodiments according to the specific test system needed. For instance, for some purposes it may be advantageous to have both a labeling reagent and a pre-treatment compound, such as a lysis-promoting compound, present in the reaction section, whilst for other applications only the pre-treatment compound is present in the reaction site while the labeling reagent is present in the sample receiving section.

The reaction section can be made of various materials such as a porous material, but also of material that partly or fully dissolves in the sample. The latter has the advantage that the user can determine visually whether or not the contents of the reaction section are contacted with the sample for an adequate time span by means of observing the disappearance of the reaction section from the backing.

The sample receiving section is optionally present at first end (A) of the backing and serves to absorb the sample and optionally withhold disturbing solid particles present in the sample. Preferably the sample receiving section is made of porous material as defined above.

The analyte detection section usually comprises one or more capture sites.

Preferably, there is a capture site present that is suitable for detection of the presence or absence of the analyte. In one embodiment this can be realized by immobilizing a capture reagent as defined above in the capture site. Said capture reagent may have a structural relationship with the analyte to be detected. Thus, when the analyte-protein-label complex that is formed by contacting the reaction site with the sample passes the capture site, binding to the capture site is not possible and there will be no signal to be observed. When there is no analyte present in the sample, there will be labeled binding particle with still accessible sites present in the sample after contact with the reaction site. Upon passage of the capture site, this labeled binding particle will bind to the capture site and a signal can be observed. Alternative embodiments are also possible. One example is by immobilizing the suitable binding particle to the capture site and incorporating a labeled analyte analogue in the reaction site. The degree to which the signal then manifests itself will then depend on the competition between analyte and labeled analyte analogue. Another example is the so-called sandwich method in which the capture site comprises a binding particle that binds to the analyte independently of whether the analyte is bound to the suitable binding particle or not.

Preferably, a second capture site is present that functions as a control site. This may be set up as an independent system by incorporating a second labeled binding particle into the reaction site and incorporating a particle that binds to said second labeled binding particle into the second capture site. This second capture site will produce a signal irrespective of whether or not an analyte is present in the sample and will this give an indication that the device functions as required. Alternatively, or in combinations with the above, the second capture site is set up as a dependent system comprising a compound that binds with the labeled binding particle. In this case the intensity of the signal in the second capture site will depend on the presence or absence of the analyte. Advantageously, this system may be used in order to obtained improved information with regard to the concentration of the analyte. Depending on the requirement of the lateral flow binding assay, also more than two capture sites having the same or different functionalities may be present. Preferably the analyte detection section and the capture site are made of porous material as defined above.

The absorption section functions to promote lateral flow through the analyte detection section. Preferably the absorption section is made of porous material as defined above.

In yet another embodiment of the first aspect of the present invention, a member is present that covers one or more of the sample receiving section, the analyte detection section, the absorption section and the reaction section. Said member, which can be made of any material, preferably a clear plastic material such as mylar, advantageously provides protection for said sections with regard to fingerprints and/or mechanical destruction and/or fumes and the like. One or more sections may be covered with a single member, however also multiple members optionally of different materials may be used.

In a second aspect of the invention, there is provided a method for the determination of an analyte in a fluid sample. The method comprises first contacting the reaction section present at second end (B) of the device of the first aspect of the present invention with the fluid sample. Temperature, stirring and time span are not stringent requirements, although it is preferred to perform this step at temperatures between 0 and 100° C., more preferably between 5 and 50° C., most preferably between 10 and 35° C. Preferably the step is carried out for a period of at least 2 seconds to 60 minutes, more preferably at least 5 seconds to 30 minutes, most-preferably 10 seconds to 10 minutes. In practice, the person skilled in the art knows what conditions to apply in order to achieve the required function of the reaction section. Secondly, the reaction section present at second end (B) of the device of the first aspect of the present invention is removed from the fluid sample. Thirdly, first end (A) of the device of the first aspect of the present invention is contacted with the fluid sample. Time span and temperature during which this operation is to take place depends on the specific type of assay that is used. The person skilled in the art is well aware of the requirements in this respect. For instance, in the case of an antibiotic assay based on a penicillin binding protein obtained from a thermophilic microorganism, such as for instance a Bacillus species such as Bacillus stearothermophilus, or a thermophilic Escherichia coli or Streptococcus species, the operation can be carried out at a temperature between 0 and 80° C., preferably between 20 and 75° C., more preferably between 35 and 70° C., most preferably between 60 and 65° C. Preferably the step is carried out for a period of at least 30 seconds to 30 minutes, more preferably at least 1 to 20 minutes, most preferably 1 to 10 minutes. Finally, the presence or absence of the analyte is detected by observing the capture site(s) of the device of the first aspect of the present invention.

In one embodiment of the second aspect of the present invention, the first step of the method comprising contacting the reaction section present at second end (B) of the device of the first aspect of the present invention with the fluid sample, is carried out such that an optimal contact between sample and reaction section is obtained. To this end, the sample is preferably placed in a container and the device of the first aspect of the present invention is placed in the sample with the reaction section of second end (B). Since sample volumes usually can be relatively small, ranging from 0.05 to 1.0 ml, preferably from 0.1 to 0.2 ml, it is normally recommended that the device is placed such that it rests in the angle between bottom and wall.

In a third aspect of the invention there is provided a kit suitable for the determination of an analyte in a fluid comprising a device according to the first aspect of the invention. The person skilled in the art knows that many applications require an assay to be performed at a constant temperature and for that reason, in one embodiment, the kit comprises a thermostatic device, with the aid of which test samples can be kept at a pre-set temperature.

In a fourth aspect of the present invention there is provided the use of a device according to any one of claims 1 to 4 for the determination of the presence or absence of an analyte in a fluid sample

### LEGEND TO THE FIGURES

FIG. 1A is a side view of the device for the detection of the presence or absence of an analyte in a fluid sample.

FIG. 1B is an exploded view of FIG. 1A. The device comprises a backing (1) with a first end (A) and a second end (B). Present on backing (1) are sample receiving section (2), an analyte detection section (3), an absorption section (5), a reaction section (6) comprising particles bound and/or conjugated to a binding particle and a member (7) covering part or all of the sample receiving section (2), the analyte detection section (3), the absorption section (5) and/or the reaction section (6). The sample receiving section (2) and the member (7) are optional. When the sample receiving section (2) is not present, the analyte detection section (3) may be placed immediately at the beginning of backing (1) at A. The sample receiving section (2), when present, is in fluid contact with the analyte detection section (3), which is in fluid contact with the absorption section (5). The analyte detection section (3) comprises at least one capture site (4a, 4b).

FIG. 2 is an embodiment of the device for the detection of the presence or absence of an analyte in a fluid sample wherein the reaction section (6) is located on the side of the backing (1) opposite to the side where the other sections (2), (3), (5) and (7) are location.

FIG. 3 is an embodiment of the device for the detection of the presence or absence of an analyte in a fluid sample wherein two reaction sections (6A and 6B) are located on both sides of the backing (1).

FIG. 4 is an embodiment of the device for the detection of the presence or absence of an analyte in a fluid sample wherein the reaction section (6) encloses end B of the backing (1).

FIG. 5 outlines to sequence of steps to be taken when performing the method of the present invention. In step (I) the fluid sample (8) is present in container (9). In step (II), the reaction section present at second end (B) of the device of the present invention is contacted with the fluid sample upon which the contents of the reaction section migrate from the reaction section to the fluid sample. In step (III), the device is turned around and first end (A) of the device is contacted with the fluid sample and the fluid sample is allowed to flow through the sample receiving section, the analyte detection section and, optionally, the absorption section. Finally, the result of the assay is determined by reading the signal of the caption site(s).

### EXAMPLES

### Example 1

### Lateral Flow Test Strips for Detection of β-Lactams in Milk

In this example a method is described for detecting the β-lactams penicillin G, amoxicillin, ampicillin, cloxacillin, cephapirin and ceftiofur in milk.

### Extraction of Antibiotic Binding Protein

A grown culture of an antibiotic sensitive microorganism, in this example Bacillus stearothermophilus (continuous culture art. # 108 Porton Products Ltd, UK) was lysed overnight at 4° C. with lysozyme, DNAse and triton X-100 in 0.1 M phosphate pH 7.0. The lysate was centrifuged for 30 minutes at approximately 1600× g (4° C.). After centrifugation the supernatant was mixed with an antibiotic affinity gel matrix. For example to prepare a 7-aminocephalosporanic acid (7-ACA) affinity gel matrix, the following method was used.

0.34 g of 7-ACA was mixed with 25 mL 0.1 M phosphate pH 7.0 (pH corrected to 7). To this solution was added 100 mL beads affigel 10® (BioRad, washed with 1 L 0.1 M phosphate pH 7.0). This was mixed gently for 2 hours at 20° C. The 7-ACA-affigel 10 was filtered and sucked off using vacuum. The 7-ACA-affigel was then washed again with 0.1 M phosphate pH 7.0 and was ready for use. The 7-ACA-affigel and the supernatant of the lysed culture was gently mixed for 3 hours at 20° C. The gel was washed with 6×500 mL 0.1 M phosphate+1 M NaCl pH 7.0.

20 mL elution buffer (0.05 M phosphate+0.5 M NaCl+0.1% triton X-100+0.8 M hydroxylamine pH 7.0) was added to the moist gel cake and gently mixed for 20 minutes at 20° C. The mixture was then centrifuged at 4° C. for 6 minutes at approximately 300 ×g.

The supernatant was dialyzed in 32 mm tubing (12-14 kD cut-off). The first dialysis was against 0.05 M phosphate+0.5 M NaCl pH 7.0 overnight at 4° C., the second up to the fifth dialysis was against 0.1 M carbonate pH 9.4 with a change of buffer every 4-6 hours. The lysate was centrifuged for 20 minutes at approximately 1000 ×g at 4° C. and concentrated in an AMICON concentrator (ultrafiltration; model # 8200, W. R. Grace and Co.) according to the manufacturer's standard operating procedure. Hereafter, the purified antibiotic binding protein was ready for conjugation.

### Conjugation of a Beta-Lactam to a Protein

The basis structure of the cephalosporins (7-ACA) was used for conjugation to Bovine Serum Albumine (BSA). A spacer between the 7-ACA and the BSA was used to obtain the best affinity and specificity for β-lactams.

40 mg of 7-ACA was added to 4 mL of 50 mM Hepes (pH 7.5) solution. After dissolving, the pH was adjusted to pH 7.0 with 1 M NaOH. Hereafter, 20 mg BSA and 40 mg bis(sulfosuccinimidyl)suberate (spacer) and an additional 2 mL of 50 mM Hepes solution were added. The mixture was shaken gently for 45 minutes at 20° C.

After mixing, the solution was dialyzed (tubing cut-off 12-14 kD) for 48 hours against PBS with three buffer changes. This dialysate was used for tube coating after dilution. An additional purification step was carried out by ultrafiltration using an AMICON concentrator (model # 8200, W. R. Grace and Co.) to eliminate the unbound 7-ACA from the 7-ACA:BSA preparation.

### Preparation of Protein-Label Complex

The PBP and a reference protein are used for conjugation to gold particles according conjugation protocols that are generally known for gold particles.

Preparation of capture line and reference line onto nitrocellulose membrane

7ACA:BSA diluted in PBS buffer and reference protein diluted in buffer are striped on a backed nitrocellulose membrane (Milipore HF90). The striping is done with the Matrix 1600 Reagent Dispensing Module of Kinematics.

### Conjugate Pad Preparation

The PBP-reference protein-gold particles are within a buffer that is adjusted to pH 7.2±0.1 and containing: 0.071 g Na₂HPO₄, 0.072 g NaCl, 1.0 g-sucrose, 0.25 g BSA, 4 mL glycerol, 50 μL Triton X-100, 45 mL H₂O, 50 μL of PBP-reference protein-gold particles with OD 12.

This gold particles containing solution is dispended with a concentration of 40 μL/cm onto a conjugate pad.

### Assembly of the Test Strip

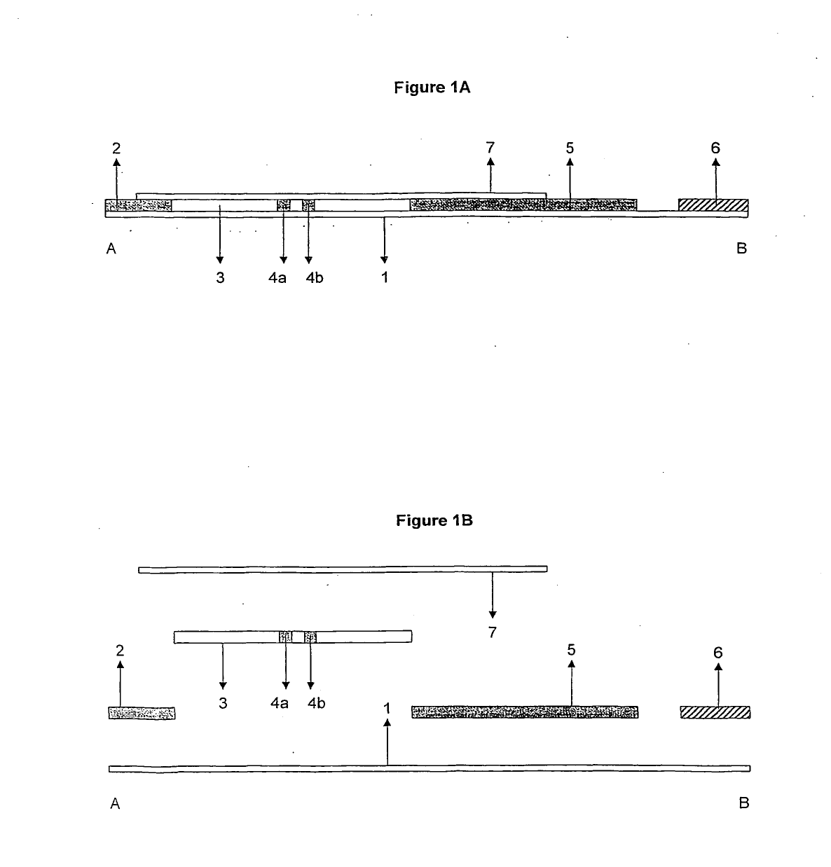

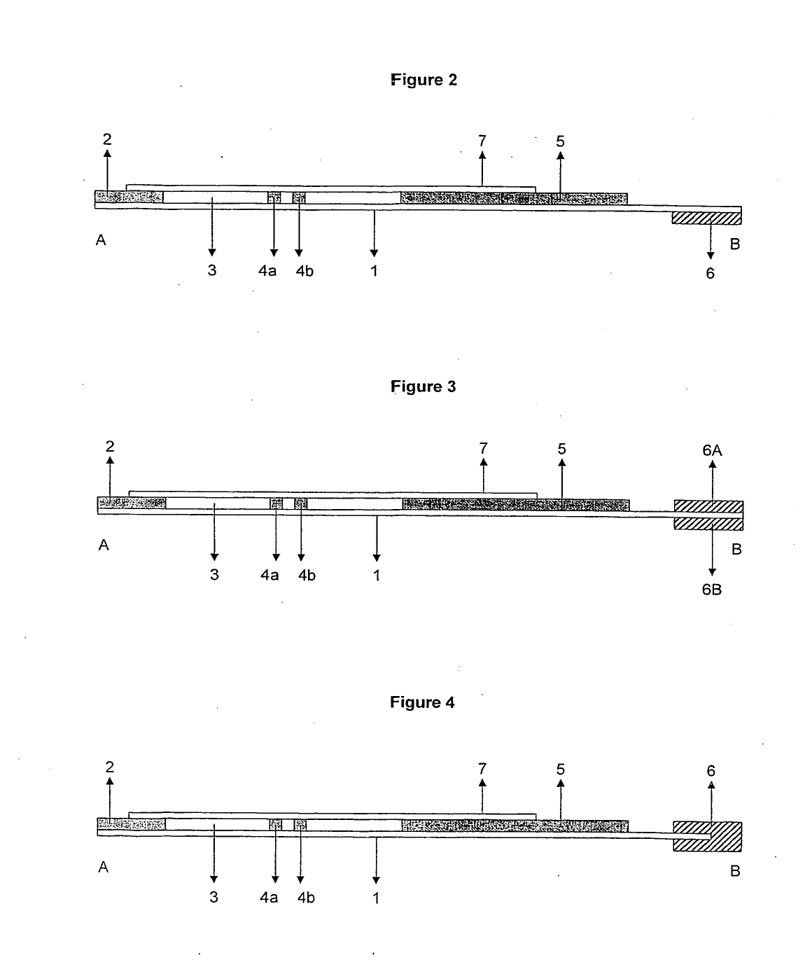

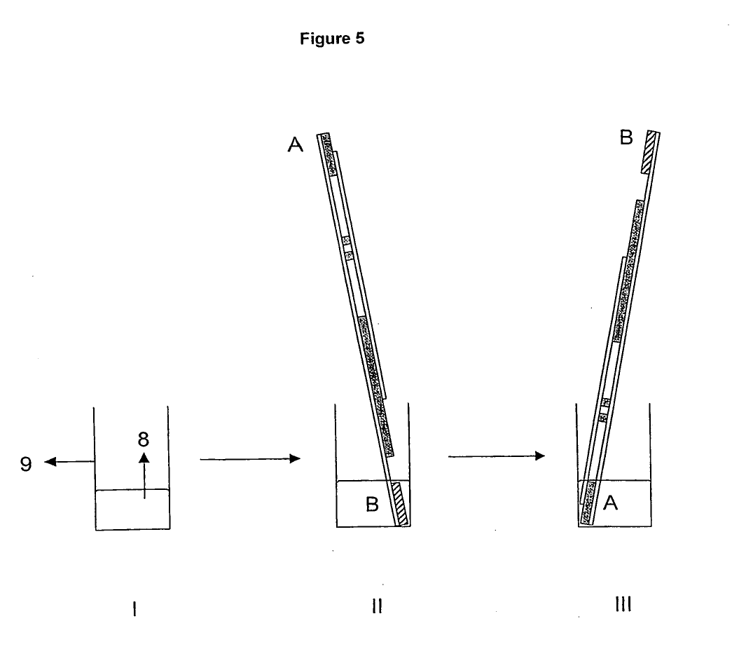

The assembly of the different membranes and pads is done by use of the Matrix 2210 Universal Laminator Module of Kinematics. After drying, the nitrocellulose was applied to the taped side of the test strip. A strip of absorbent paper (Alistrom 222; 4 cm) was applied just above and touching the nitrocellulose, at the positions indicated by the zones in FIG. 1. The conjugate pad was applied, after drying, the to position on the backing as is also indicated by FIG. 1. The assembled sheets of membranes are cut into 0.5 cm strips by the use of the Matrix 2360 Programmable Shear of Kinematics.

### Test-Performance (Sequential Assay)

0.1 mL of milk sample was added to an empty reaction vial and put into an incubator (SRP incubator). The test strip was put into the vial with the reagent region making contact with the milk sample; the strip was placed in the corner site of the bottom. After incubation of 5 minutes at 64° C., the test strip was taken out and inversely replaced into the vial, also at the corner site of the bottom. After 10 minutes of incubation at 64° C., the test strip was taken out and the signal was read visually. A darker or equal intensity of the lower capture line, compared to the higher placed reference line indicates that the milk sample does not contain residues of the β-lactams beyond the sensitivity level as indicated in the Table below. A lighter intensity of the capture line compared to the reference line indicates that β-lactam residues are present above the sensitivity level indicated in the Table below.

This test device according to this example is sensitive for β-lactams as indicated in the Table below.

| Antibiotic | Sensitivity (ppb) |
|---|---|
| Penicillin G | 4 |
| Amoxicillin | 4 |
| Ampicillin | 4 |
| Cloxacillin | 60 |
| Cephapirin | 4 |
| Ceftiofur | 4 |

1. A device for the detection of the presence or absence of an analyte in a fluid sample comprising a backing with a first end (A) and a second end (B) and consecutively going from first end (A) to second end (B):
(a) a sample receiving section attached to one side of the backing;
(b) an analyte detection section attached to said one side of the backing comprising a capture site;
(c) an absorption section attached to said one side of the backing;
(d) a reaction section;
wherein the sample receiving section is in fluid contact with the analyte detection section, which is in fluid contact with the absorption section and wherein there is no fluid contact between the reaction section and any of the other sections.

2. Device according to claim 1 wherein the reaction section comprises
(a) particles bound and/or conjugated to a binding particle, and/or
(b) pretreatment compounds.

3. Device according to claim 1 further comprising a member covering part or all of the sample receiving section, the analyte detection section, the absorption section and/or the reaction section.

4. Device according to claim 1 wherein the reaction section is located on said one side of the backing or opposite to said one side of the backing or enclosing second end (B) of the backing.

5. A method for detecting an analyte in a fluid sample comprising providing a device as in claim 1, and thereafter performing the steps of:
(a) contacting the reaction section present at the second end (B) of the device with the fluid sample for a period of at least 10 seconds to 10 minutes;
(b) removing the reaction section present at the second end (B) of the device from the fluid sample;
(c) contacting the first end (A) of the device claim 1 with the fluid sample for a period of at least 1 to 10 minutes; and
(d) detecting the analyte by observing the capture site of the device claim 1.

6. Method according to claim 5 wherein step (a) comprises stirring the device.

7. Method according to claim 5 wherein the analyte to be determined is a β-lactam antibiotic.

8. Method according to claim 5 wherein the fluid in which an analyte is to be determined is a fluid obtainable from an animal or human body.

9. Kit suitable for the determination of an analyte in a fluid comprising a device according to claim 1 and optionally a thermostatic device, with the aid of which test samples can be kept at a pre-set temperature.

10. Use of a device according to claim 1 for the determination of the presence or absence of an analyte in a fluid sample
